(19) United States
(12) Patent Application Publication
(10) Pub. No.: US 2026/0086025 A1
(43) Pub. Date: Mar. 26, 2026

(54) Flame Atomic Absorption Photometer

(71) Applicant: SHIMADZU CORPORATION, Kyoto-shi (JP)

(72) Inventors: Nobuyuki IWAI, Kyoto-shi (JP); Tomoari KOBAYASHI, Kyoto-shi (JP); Takuya KIMOTO, Kyoto-shi (JP); Osuke KOBAYASHI, Kyoto-shi (JP)

(21) Appl. No.: 19/123,205

(22) PCT Filed: Jun. 2, 2023
(86) PCT No.: PCT/JP2023/020679

§ 371 Date: Apr. 22, 2025

(30) Foreign Application Priority Data
Oct. 31, 2022 (JP) 2022-174434

(51) Int. Cl. G01N 21/3504; G01N 21/33; G01N 33/00

(52) U.S. Cl. CPC G01N 21/3504; G01N 21/33; G01N 33/0013

(57) ABSTRACT

The present invention provides a flame atomic absorption photometer including a burner 110 configured to form a flame by burning a mixture of a mixed gas of a fuel gas and a combustion-assisting gas and a nebulized sample liquid, flame light detection units 181 and 182 configured to detect light radiated from the flame, and a ceased flame determination unit 162 configured to determine that the flame has ceased when the intensity of the light detected by the flame light detection unit is lower than a predetermined threshold value, wherein the flame light detection unit is configured to selectively detect light having a wavelength of 290 nm or more and 330 nm or less.

### TECHNICAL FIELD

The present invention relates to a flame atomic absorption photometer.

### BACKGROUND ART

In the flame atomic absorption photometer, a sample liquid nebulized by a nebulizer and combustion gas are mixed in a chamber, and the mixed gas is blown out from a slit opening of a burner head, and burned to form a flame. When the components in the sample are atomized in the flame and the flame containing the atomized sample components is irradiated with light, only light having a specific wavelength corresponding to the atoms (elements) is absorbed. Therefore, by measuring the absorption of light by the sample atoms, the elements in the sample can be identified and quantified.

The combustion gas for forming the flame is usually a mixed gas of a fuel gas of a hydrocarbon such as acetylene (C₂H₂) and a combustion-assisting gas of such as air or dinitrogen monoxide (N₂O). When the combustion gas is normally combusted, the combustion speed and the flow speed of the gas blown out from the burner head are balanced, so that the flame is stably formed at a position slightly above the upper end of the burner head.

However, when the balance between the combustion speed and the gas flow speed is lost for some reason, or when wind blows in from the outside, the flame may be extinguished (that is, the flame may cease) at an undesired timing. Therefore, some conventional flame atomic absorption photometers have a function of providing an optical sensor in the vicinity of a flame to always monitor the intensity of light (flame light) emitted from the flame, and automatically extinguishing the fire and stopping the supply of combustion gas when the intensity is less than the light intensity at the time of normal combustion (see, for example, Patent Literature 1).

### CITATION LIST

### Patent Literature

Patent Literature 1: JP 2010-127812 A

### SUMMARY OF INVENTION

### Technical Problem

However, even in the flame atomic absorption photometer including the optical sensor for monitoring flame light as described above, depending on the environment of use, it may erroneously determine that the flame is standing normally although the light intensity of the flame is actually less than the light intensity at the time of normal combustion due to stray light such as sunlight or indoor illumination incident on the optical sensor.

Furthermore, when the balance between the combustion speed of the burner and the flow speed of the gas blown out from the burner head collapses, the flame enters the inside of the burner, and backfire, which is unstable combustion, may occur. Conventionally, in order to ensure the safety of the flame atomic absorption photometer, various mechanisms for preventing such backfire have been proposed, but there is still room for improvement.

Furthermore, when the flow rate of the combustion-assisting gas is too small with respect to the flow rate of the fuel gas, incomplete combustion may occur, and a toxic gas such as carbon monoxide may be generated, or the temperature of the flame may decrease, and atomization of the sample may be insufficient. Therefore, a mechanism for reliably detecting incomplete combustion has been required.

Furthermore, in the flame atomic absorption photometer, when the combustion state of the flame becomes unstable temporarily or continuously, soot is generated and accumulates on the burner head. This soot is a cause of impairing the stability of the flame, and thus needs to be appropriately removed. However, conventionally, since the user checked the accumulation status of soot by directly viewing the burner head, there is a case where the accumulation status of soot cannot be appropriately grasped.

The present invention has been made in view of these points, and an object of the present invention is to enable a flame atomic absorption photometer to accurately detect an abnormality related to stability of a combustion state of a flame. More specifically, a first object is to provide a flame atomic absorption photometer capable of reliably determining whether a flame continues to burn normally. Furthermore, a second object is to enable a flame atomic absorption photometer to detect the occurrence of backfire in advance. Furthermore, a third object is to enable a flame atomic absorption photometer to reliably detect occurrence of incomplete combustion. Furthermore, a fourth object is to enable a user to reliably grasp the accumulation status of soot on the burner head in the flame atomic absorption photometer.

### Solution to Problem

A flame atomic absorption photometer according to a first mode of the present invention made to solve the above problems includes:
a burner configured to form a flame by burning a mixture of a mixed gas of a fuel gas and a combustion-assisting gas and a nebulized sample liquid;
a flame light detection unit configured to detect light radiated from the flame; and
a ceased flame determination unit configured to determine that the flame has ceased when the intensity of the light detected by the flame light detection unit is lower than a predetermined threshold value; in which
   the flame light detection unit is configured to selectively detect light having a wavelength of 290 nm or more and 330 nm or less.

A flame atomic absorption photometer according to a second mode of the present invention made to solve the above problems includes:
a burner configured to form a flame by burning a mixture of a mixed gas of a fuel gas and a combustion-assisting gas and a nebulized sample liquid;
a flame light detection unit configured to detect light radiated from the flame and selectively detect light having a wavelength of 290 nm or more and 330 nm or less;
a swan band light detection unit configured to selectively detect light of a C₂ swan band among light radiated from the flame; and
a backfire sign determination unit configured to determine that there is a sign of backfire when a ratio of a light intensity detected by the swan-band light detection unit to a light intensity detected by the flame light detection unit falls below a predetermined threshold value.

A flame atomic absorption photometer according to a third mode of the present invention made to solve the above problems includes:
a burner configured to form a flame by burning a mixture of a mixed gas of a fuel gas and a combustion-assisting gas and a nebulized sample liquid;
a flame light detection unit configured to detect light radiated from the flame and selectively detect light having a wavelength of 290 nm or more and 330 nm or less;
a bright flame detection unit configured to selectively detect light having a wavelength of 800 nm or more and 1100 nm or less among the light radiated from the flame; and
an incomplete combustion determination unit configured to determine that incomplete combustion has occurred when a ratio of a light intensity detected by the bright flame detection unit to a light intensity detected by the flame light detection unit exceeds a predetermined threshold value.

A flame atomic absorption photometer according to a fourth mode of the present invention made to solve the above problems includes:
a burner configured to form a flame by burning a mixture of a mixed gas of a fuel gas and a combustion-assisting gas and a nebulized sample liquid;
a flame light detection unit configured to detect light radiated from the flame and selectively detect light having a wavelength of 290 nm or more and 330 nm or less;
a bandpass filter configured to selectively transmit light having a wavelength of 800 nm or more and 1100 nm or less;
an image sensor including a plurality of light detection elements arranged two-dimensionally and configured to receive light radiated from the burner and passing through the bandpass filter;
a soot accumulation determination unit configured to determine that soot is accumulated on the burner, by obtaining, for each of the plurality of light detection elements, a ratio of a light intensity detected by the light detection element to a light intensity detected by the flame light detection unit, and when a predetermined number or more of the plurality of light detection elements have the ratio exceeding a predetermined threshold value; and
a notification unit configured to, when the soot accumulation determination unit determines that soot is accumulated on the burner, notify a user of the determination.

### Advantageous Effects of Invention

With the flame atomic absorption photometer according to the first mode, it is possible to reliably determine whether or not the flame continues to burn normally. With the flame atomic absorption photometer according to the second mode, the occurrence of backfire can be detected in advance. With the flame atomic absorption photometer according to the third mode, occurrence of incomplete combustion can be reliably detected. With the flame atomic absorption photometer according to the fourth mode, the user can reliably grasp the accumulation status of soot. Therefore, with the flame atomic absorption photometers according to the first to fourth modes, it is possible to accurately detect an abnormality related to the stability of the combustion state of the flame.

### BRIEF DESCRIPTION OF DRAWINGS

FIG. 1 A view showing a configuration of a main part of a flame atomic absorption photometer according to a first embodiment of the present invention.

FIG. 2 A view showing a configuration of a main part of a flame atomic absorption photometer according to a second embodiment of the present invention.

FIG. 3 A view showing a configuration of a main part of a flame atomic absorption photometer according to a third embodiment of the present invention.

FIG. 4 A view showing a configuration of a main part of a flame atomic absorption photometer according to a fourth embodiment of the present invention.

FIG. 5 A view showing a configuration of a main part of a flame atomic absorption photometer according to a fifth embodiment of the present invention.

### DESCRIPTION OF EMBODIMENTS

### First Embodiment

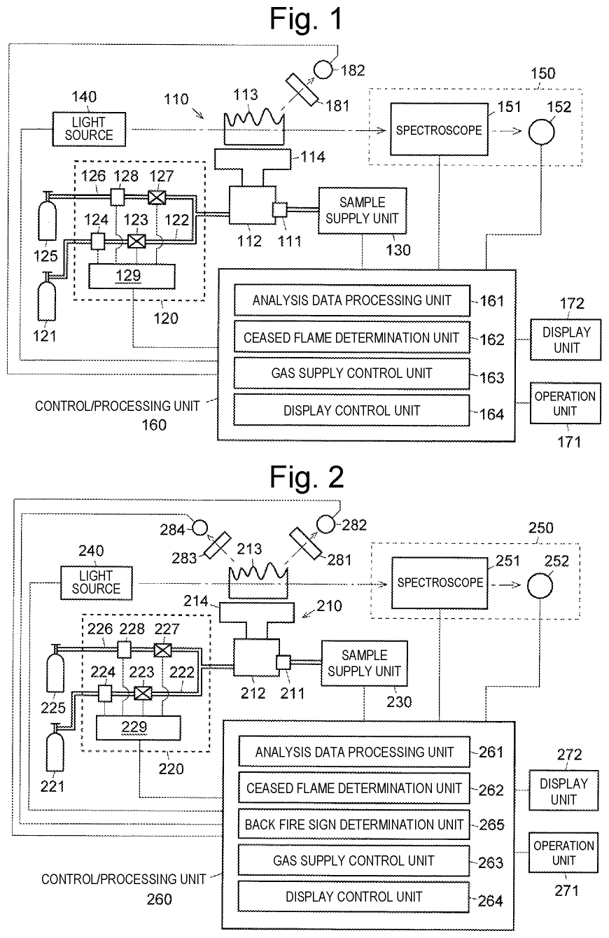

Hereinafter, a flame atomic absorption photometer according to a first embodiment of the present invention will be described with reference to FIG. 1. FIG. 1 is a configuration diagram of a main part of a flame atomic absorption photometer according to the present embodiment. The flame atomic absorption photometer includes a burner 110, a gas supply unit 120, a sample supply unit 130, a light source 140, a spectroscopic unit 150, and a control/processing unit 160.

The burner 110 includes a nebulizer 111 that nebulizes a sample liquid, a chamber 112 that mixes the nebulized sample liquid with combustion gas, and a burner head 114 that blows the mixed gas upward and burns the gas to form a flame 113. Note that the burner 110 is provided with an ignition unit (not illustrated) that ignites (lights) the gas. A mixed gas of acetylene as a fuel gas and air or dinitrogen monoxide as a combustion-assisting gas is supplied from the gas supply unit 120 to the chamber 112 as a combustion gas.

The gas supply unit 120 includes a fuel gas supply pipe 122 that guides fuel gas from a fuel gas source 121 such as a gas cylinder to the burner 110, a fuel gas pipe on-off valve 123 and a fuel gas flow rate adjusting valve 124 provided on the fuel gas supply pipe 122, a combustion-assisting gas supply pipe 126 that guides combustion-assisting gas from a combustion-assisting gas source 125 such as a gas cylinder or an air compressor to the burner 110, a combustion-assisting gas pipe on-off valve 127 and a combustion-assisting gas flow rate adjusting valve 128 provided on the combustion-assisting gas supply pipe 126, and a valve drive unit 129 that drives these valves 123, 124, 127, and 128.

The light source 140 is disposed on a side of a region where flame 113 is formed (hereinafter, referred to as flame forming region). The spectroscopic unit 150 includes a spectroscope 151 and a photodetector 152, and is disposed at a position facing the light source 140 across the flame forming region. Light having an emission-line spectrum including a resonance line of a target element is emitted from the light source 140, and this light passes through atomic vapor in the flame forming region. The light that has passed through the atomic vapor is dispersed by the spectroscope 151, and light having a specific wavelength corresponding to an emission-line (usually a resonance line) having the highest absorbance by the target element is extracted. The light having the specific wavelength is introduced into the photodetector 152, and a detection signal corresponding to the intensity of incident light is output. The detection signal is amplified by an amplifier (not illustrated), converted into a digital signal by an A/D converter (not illustrated), and input to the control/processing unit 160. An analysis data processing unit 161, which is a functional block provided in the control/processing unit 160, calculates the absorbance for the specific wavelength light on the basis of the digital signal, and further performs predetermined arithmetic processing to perform quantitative analysis.

The control/processing unit 160 is mainly composed of a computer including a CPU, a memory, and the like, performs various arithmetic processing, and outputs a control signal for controlling the operation of each unit. In addition to the analysis data processing unit 161 described above, the control/processing unit 160 includes a ceased flame determination unit 162, a gas supply control unit 163, and a display control unit 164 as functional blocks. Furthermore, an operation unit 171 such as a keyboard and a display unit 172 such as a liquid crystal display are connected to the control/processing unit 160, and an instruction from the user is input to the control/processing unit 160 via the operation unit 171, and an analysis result or the like are displayed on the display unit 172.

Further, an OH-derived light detecting optical sensor 182 which is an optical sensor for detecting light derived from OH radicals (hereinafter, simply referred to as OH) in the flame 113 is disposed in the vicinity of the flame forming region, and an OH-derived light transmitting bandpass filter 181 which is a bandpass filter for selectively transmitting light having a wavelength of around 310 nm is disposed between the OH-derived light detecting optical sensor 182 and the flame forming region (the OH-derived light transmitting bandpass filter 181 and the OH-derived light detecting optical sensor 182 correspond to a flame light detection unit in the present invention). Here, around 310 nm is, for example, a range of 290 nm to 330 nm, desirably 300 nm to 320 nm. The OH-derived light detecting optical sensor 182 desirably receives light from the entire region of the flame 113, but may receive light from only a part of the flame 113. Note that in FIG. 1, for convenience of drawing, the OH-derived light detecting optical sensor 182 is disposed obliquely above the flame 113, but the position where the OH-derived light detecting optical sensor 182 is provided is not limited to this (hereinafter, the same applies to second to fifth embodiments). As the OH-derived light detecting optical sensor 182, for example, a phototransistor can be suitably used, but the present invention is not limited to this, and any device such as a photodiode, a photoelectric tube, or a photomultiplier tube may be used.

Combustion flames of hydrocarbons such as acetylene include emission spectrum derived from OH. The emission spectrum derived from OH exists in a plurality of regions in the ultraviolet region, but a band spectrum in the 310 nm band (3064 Å system) has high intensity, high transmittance of the optical element, and high detection sensitivity by a general optical sensor. On the other hand, ambient light such as sunlight, an incandescent lamp, a fluorescent lamp, or a white LED, which affect as stray light, all have low intensity in the 310 nm band. Therefore, with the flame atomic absorption photometer according to the present embodiment, since the OH-derived light transmitting bandpass filter 181 that selectively transmits light having a wavelength of around 310 nm as described above is provided in front of the OH-derived light detecting optical sensor 182, the OH-derived light detecting optical sensor 182 can be prevented from being affected by stray light.

The light radiated from the flame 113 and passing through the OH-derived light transmitting bandpass filter 181 is incident on the OH-derived light detecting optical sensor 182, and a detection signal corresponding to the incident light intensity is output from the OH-derived light detecting optical sensor 182. This detection signal is amplified by an amplifier (not illustrated), converted into a digital signal by an A/D converter (not illustrated), and input to the ceased flame determination unit 162. The ceased flame determination unit 162 compares the intensity of the digital signal with a predetermined threshold value T₁, and determines that the flame 113 has ceased when the digital signal is below the threshold value T₁. Note that the threshold value T₁ may be set at a stage before the present device is delivered to the user or at a stage of installation of the present device, or may be set by the user.

When the ceased flame determination unit 162 determines that the flame 113 has ceased, the gas supply control unit 163 controls the valve drive unit 129 to close the fuel gas pipe on-off valve 123 and the combustion-assisting gas pipe on-off valve 127.

After the fuel gas pipe on-off valve 123 and the combustion-assisting gas pipe on-off valve 127 are closed, the display control unit 164 controls the display unit 172 to display a predetermined message on the screen, thereby notifying the user that the gas supply has been stopped due to the flame cessation. Note that a message notifying the user that the flame 113 has ceased may be displayed on the screen of the display unit 172 before or simultaneously with closing of the fuel gas pipe on-off valve 123 and the combustion-assisting gas pipe on-off valve 127. Alternatively, only the fuel gas pipe on-off valve 123 and the combustion-assisting gas pipe on-off valve 127 may be closed without performing such notification. Furthermore, the configuration may be such that the fuel gas pipe on-off valve 123 and the combustion-assisting gas pipe on-off valve 127 are not closed, and only the notification that the flame 113 has ceased is performed.

### Second Embodiment

Next, a flame atomic absorption photometer according to a second embodiment of the present invention will be described with reference to FIG. 2. FIG. 2 is a configuration diagram of a main part of a flame atomic absorption photometer according to the present embodiment. Note that in the present embodiment, the same or corresponding components as those illustrated in FIG. 1 are denoted by the same reference numerals in the last two digits, and the description of such components is appropriately omitted.

The flame atomic absorption photometer according to the present embodiment includes, in addition to the same configuration as the flame atomic absorption photometer according to the first embodiment, a C₂-derived light transmitting bandpass filter 283 and a C₂-derived light detecting optical sensor 284 provided in the vicinity of a flame forming region, and a backfire sign determination unit 265 that is a functional block provided in a control/processing unit 260. Among them, the C₂-derived light transmitting bandpass filter 283 and the C₂-derived light detecting optical sensor 284 correspond to a swan-band light detection unit in the present invention. Furthermore, in the present embodiment, a gas supply control unit 263 corresponds to a backfire avoidance unit in the present invention.

Normally, in a burner 210 of the flame atomic absorption photometer, the ratio of the combustion-assisting gas flow rate to the fuel gas flow rate (that is, the air-fuel ratio) is set to be significantly smaller than the stoichiometric air-fuel ratio (the air-fuel ratio when the combustion-assisting gas and the fuel gas in the combustion gas react with each other without excess or deficiency and the combustion speed becomes the highest). However, when the air-fuel ratio approaches the stoichiometric air-fuel ratio for some reason, the combustion reaction is promoted, the region of an outer flame portion where the emission spectrum derived from OH is remarkable expands in a flame 213, and the region of an inner flame portion including the emission spectrum derived from C₂ (diatomic carbon) in the transient process of the reaction decreases. In this state, when the air-fuel ratio further increases and the combustion speed becomes excessive with respect to the supply speed of the combustion gas, the flame 213 cannot be continuously formed outside the burner 210 and enters the inside of the burner 210, and backfire that is unstable combustion occurs. The flame atomic absorption photometer according to the present embodiment has a function of detecting the light from the outer flame portion and the light from the inner flame portion and detecting a sign of backfire based on an intensity ratio between the light from the outer flame portion and the light from the inner flame portion in order to prevent the occurrence of such backfire.

As the emission spectrum derived from C₂, a band spectrum of a C₂ swan system is known. The C₂-derived light transmitting bandpass filter 283 in the present embodiment selectively transmits light in a wavelength band of a band spectrum of the C₂ swan system. The C₂ swan system has a plurality of band spectra in the visible light region, and emission around 517 nm is particularly remarkable. Therefore, it is desirable that the C₂-derived light transmitting bandpass filter 283 in the present embodiment selectively transmits light having a wavelength of 507 nm to 527 nm (desirably 512 nm to 522 nm). However, the transmission wavelength range by the C₂-derived light transmitting bandpass filter 283 is not limited to this, and light in a wavelength range of other band spectra included in the C₂ swan system, that is, light of 464 nm to 484 nm (desirably 469 nm to 479 nm) or 554 nm to 574 nm (desirably 559 nm to 569 nm) may be selectively transmitted. The C₂-derived light detecting optical sensor 284 is a sensor that detects light radiated from the flame 213 and passing through the C₂-derived light transmitting bandpass filter 283. As the C₂-derived light detecting optical sensor 284, a phototransistor can be suitably used, but the present invention is not limited to this, and any device such as a photodiode, a photoelectric tube, or a photomultiplier tube may be used. Furthermore, the C₂-derived light detecting optical sensor 284 may receive light from the entire region of the flame 213, but it is most effective to receive only light from a lower region of the flame 213 where C₂ is localized. Note that in FIG. 2, for convenience of drawing, the C₂-derived light detecting optical sensor 284 is disposed obliquely above the flame 213, but the position where the C₂-derived light detecting optical sensor 284 is provided is not limited to this.

The light radiated from the flame 213 and passing through the C₂-derived light transmitting bandpass filter 283 is incident on the C₂-derived light detecting optical sensor 284, and a detection signal corresponding to the incident light intensity is output from the C₂-derived light detecting optical sensor 284. This detection signal is amplified by an amplifier (not illustrated), converted into a digital signal by an A/D converter (not illustrated), and input to the backfire sign determination unit 265 (this signal is hereinafter referred to as “C₂-derived light detection signal”). On the other hand, the detection signal from the OH-derived light detecting optical sensor 282 is amplified and digitally converted as in the first embodiment, and then input to the control/processing unit 260 (this signal is hereinafter referred to as “OH-derived light detection signal”). The OH-derived light detection signal is input to the ceased flame determination unit 262 as in the first embodiment and is used for determining whether or not the flame 213 has ceased, and is also input to the backfire sign determination unit 265. The backfire sign determination unit 265 divides the intensity of the C₂-derived light detection signal by the intensity of the OH-derived light detection signal (that is, obtains the ratio of the C₂-derived light detection signal to the OH-derived light detection signal), and compares the value with a predetermined threshold value T₂. When the value obtained by dividing the intensity of the C₂-derived light detection signal by the intensity of the OH-derived light detection signal is less than the threshold value T₂, it is determined that there is a sign of backfire. Note that the threshold value T₂ may be set at a stage before the present device is delivered to the user or at a stage of installation of the present device, or may be set by the user. In this manner, by determining the presence or absence of a sign of backfire on the basis of the ratio of the C₂-derived light detection signal to the OH-derived light detection signal, it is possible to cancel a change in the intensity of the C₂-derived light due to the fluctuation of the flame 213 and perform accurate determination.

When the backfire sign determination unit 265 determines that there is an indication of backfire, the gas supply control unit 263 controls a valve drive unit 229 so that the air-fuel ratio in the combustion gas supplied to the burner 210 decreases. Specifically, the opening degree of the fuel gas flow rate adjusting valve 224 is gradually increased, the opening degree of the combustion-assisting gas flow rate adjusting valve 228 is gradually decreased, or both of them are performed until the backfire sign determination unit 265 determines that there is no sign of backfire (that is, until it is determined that the ratio of the C₂-derived light detection signal to the OH-derived light detection signal is equal to or greater than the threshold value T₂).

After adjusting the opening degree of the fuel gas flow rate adjusting valve 224 and/or the opening degree of the combustion-assisting gas flow rate adjusting valve 228 (hereinafter, it is simply referred to as gas flow rate adjustment), a display control unit 264 controls a display unit 272 to display a predetermined message on the screen, thereby notifying the user that the gas flow rate has been adjusted because there is a sign of backfire. Note that a message notifying that there is a sign of backfire may be displayed on the screen of the display unit 272 simultaneously with the gas flow rate adjustment or before the gas flow rate adjustment. Alternatively, only the gas flow rate adjustment may be performed without performing such notification. Furthermore, without adjusting the gas flow rate, only notification that there is a sign of backfire may be performed.

Alternatively, a mechanism for reducing the combustion speed of the flame 213 by reintroducing the exhaust gas from the burner 210 into the burner 210 may be provided in addition to or instead of the gas flow rate adjustment as described above when the backfire sign determination unit 265 determines that there is an indication of backfire. A flame atomic absorption photometer (flame atomic absorption photometer according to a third embodiment of the present invention) having such a mechanism will be described below.

### Third Embodiment

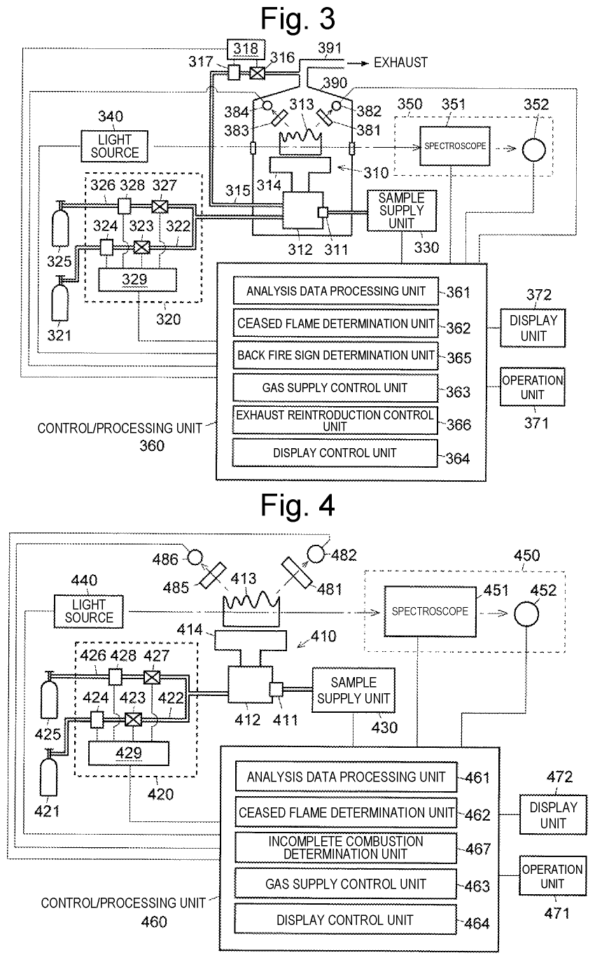

FIG. 3 is a configuration diagram of a main part of the flame atomic absorption photometer according to the third embodiment of the present invention. Note that in the present embodiment, the same or corresponding components as those described in the first embodiment or the second embodiment are denoted by the same reference numerals in the last two digits, and the description of such components is appropriately omitted.

The flame atomic absorption photometer according to the present embodiment includes, in addition to the same configuration as the flame atomic absorption photometer according to the second embodiment, an exhaust reintroduction pipe 315 for returning some of the exhaust gas generated from a burner 310 to the burner 310, an on-off valve (hereinafter, referred to as exhaust on-off valve 316) and a flow rate adjusting valve (hereinafter, referred to as exhaust flow rate adjusting valve 317) provided on the exhaust reintroduction pipe 315, an exhaust valve drive unit 318 that drives these valves 316 and 317, and an exhaust reintroduction control unit 366 that is a functional block provided in a control/processing unit 360 and controls the exhaust valve drive unit 318. The exhaust reintroduction pipe 315, the exhaust on-off valve 316, the exhaust flow rate adjusting valve 317, the exhaust valve drive unit 318, and the exhaust reintroduction control unit 366 correspond to an exhaust introduction unit in the present invention. The exhaust reintroduction pipe 315 is a pipe branched from an exhaust pipe 391 for discharging exhaust air to the outside from a burner chamber 390 in which the burner 310 is housed, and its tip is connected to a chamber 312 of the burner 310. Note that the burner chamber 390 and the exhaust pipe 391 are also provided in the flame atomic absorption photometers according to the first embodiment and the second embodiment, but are not illustrated in these embodiments for the sake of simplicity (the same applies to a fourth embodiment and a fifth embodiment described later).

In the flame atomic absorption photometer according to the present embodiment, when a backfire sign determination unit 365 determines that there is a sign of backfire, the exhaust reintroduction control unit 366 controls the exhaust valve drive unit 318 to recirculate the exhaust gas generated in the burner 310 to the chamber 312 of the burner 310. Specifically, when it is determined that there is a sign of backfire, the exhaust on-off valve 316 is first opened, and further, the opening degree of the exhaust flow rate adjusting valve 317 is gradually increased until it is determined that there is no sign of backfire in the backfire sign determination unit 365. As a result, the combustion speed of the flame 313 is suppressed, and the occurrence of backfire can be avoided.

Note that in the flame atomic absorption photometer according to the present embodiment, when it is determined that there is a sign of backfire by the backfire sign determination unit 365, in addition to the recirculation of the exhaust gas as described above, the flow rate of the combustion-assisting gas and/or the fuel gas may be adjusted as in the second embodiment. Since the method for determining a backfire sign and the method for adjusting the flow rate of the combustion-assisting gas and/or the fuel gas in the present embodiment are similar to those in the second embodiment, the description is omitted here.

### Fourth Embodiment

Next, a flame atomic absorption photometer according to the fourth embodiment of the present invention will be described with reference to FIG. 4. FIG. 4 is a configuration diagram of a main part of a flame atomic absorption photometer according to the present embodiment. Note that in the present embodiment, the same or corresponding components as those described in the first embodiment are denoted by the same reference numerals in the last two digits, and the description of such components is appropriately omitted.

The flame atomic absorption photometer according to the present embodiment includes, in addition to the same configuration as the flame atomic absorption photometer according to the first embodiment, a bright flame light transmitting bandpass filter 485 and a bright flame light detection optical sensor 486 provided in the vicinity of a flame forming region, and an incomplete combustion determination unit 467 which is a functional block provided in a control/processing unit 460. Among them, the bright flame light transmitting bandpass filter 485 and the bright flame light detection optical sensor 486 correspond to a bright flame detection unit in the present invention. Furthermore, in the present embodiment, a gas supply control unit 463 corresponds to an incomplete combustion cancellation unit in the present invention.

The bright flame light transmitting bandpass filter 485 is a bandpass filter that selectively transmits light (bright flame) radiated from soot in a flame 413, and specifically, selectively transmits light in the all or part of wavelength range of 800 nm to 1100 nm. The bright flame light detection optical sensor 486 is a sensor that detects light radiated from the flame 413 and passing through the bright flame light transmitting bandpass filter 485. As the bright flame light detection optical sensor 486, a phototransistor can be suitably used, but the present invention is not limited to this, and any device such as a photodiode, a photoelectric tube, or a photomultiplier tube may be used. Furthermore, the bright flame light detection optical sensor 486 desirably receives light from the entire region of the flame 413, but may receive light from only a part of the flame 413. Note that in FIG. 4, for convenience of drawing, the bright flame light detection optical sensor 486 is disposed obliquely above the flame 413, but the position where the bright flame light detection optical sensor 486 is provided is not limited to this.

In a burner 410, when the air-fuel ratio becomes excessively small, incomplete combustion occurs, and soot is generated in the flame 413. Bright flame, which is light radiated from soot heated to a high temperature, has a continuous spectrum with high brightness and thermal equilibrium. The continuous spectrum generated from soot under the temperature of the flame 413 (to 3000 K) has little energy in the 310 nm band in which the above-described OH emits light. Therefore, an OH-derived light detecting optical sensor 482 provided with an OH-derived light transmitting bandpass filter 481 that selectively transmits the 310 nm band is not affected by the bright flame, and it is possible to monitor whether or not the flame 413 continues to burn.

The light radiated from the flame 413 and passing through the bright flame light transmitting bandpass filter 485 is incident on the bright flame light detection optical sensor 486, and a detection signal corresponding to the incident light intensity is output from the bright flame light detection optical sensor 486. This detection signal is amplified by an amplifier (not illustrated), converted into a digital signal by an A/D converter (not illustrated), and input to the incomplete combustion determination unit 467 (this signal is hereinafter referred to as “bright flame detection signal”). On the other hand, the detection signal from the OH-derived light detecting optical sensor 482 is amplified and digitally converted as in the first embodiment, and then input to the control/processing unit 460 (this signal is hereinafter referred to as “OH-derived light detection signal”). The OH-derived light detection signal is input to a ceased flame determination unit 462 as in the first embodiment and is used for determining whether or not the flame 413 has ceased, and is also input to the incomplete combustion determination unit 467. The incomplete combustion determination unit 467 divides the intensity of the bright flame detection signal by the intensity of the OH-derived light detection signal (that is, obtains the ratio of the bright flame detection signal to the OH-derived light detection signal), and compares the value with a predetermined threshold value T₄. When the value obtained by dividing the intensity of the bright flame detection signal by the intensity of the OH-derived light detection signal exceeds the threshold value T₄, it is determined that incomplete combustion has occurred in the burner 410. Note that the threshold value T₄ may be set at a stage before the present device is delivered to the user or at a stage of installation of the present device, or may be set by the user. In this manner, by determining whether or not incomplete combustion has occurred based on the ratio of the bright flame detection signal to the OH-derived light detection signal, it is possible to cancel a change in the intensity of the bright flame light due to the fluctuation of the flame 413 and to perform accurate determination.

When the incomplete combustion determination unit 467 determines that incomplete combustion has occurred, the gas supply control unit 463 controls a valve drive unit 429 to increase the air-fuel ratio in the combustion gas supplied to the burner 410. Specifically, the opening degree of a fuel gas flow rate adjusting valve 424 is gradually reduced, the opening degree of a combustion-assisting gas flow rate adjusting valve 428 is gradually increased, or both of them are performed until the incomplete combustion determination unit 467 determines that the incomplete combustion has not occurred (that is, until it is determined that the ratio of the bright flame detection signal to the OH-derived light detection signal is equal to or less than the threshold value).

After adjusting the fuel gas flow rate adjusting valve 424 and/or the combustion-assisting gas flow rate adjusting valve 428 (hereinafter, it is simply referred to as gas flow rate adjustment), a display control unit 464 controls a display unit 472 to display a predetermined message on the screen, thereby notifying the user that the gas flow rate has been adjusted due to the occurrence of the incomplete combustion. Note that a message notifying the user that the incomplete combustion has occurred may be displayed on the screen of the display unit 472 simultaneously with the gas flow rate adjustment or before the gas flow rate adjustment. Alternatively, only the gas flow rate adjustment may be performed without performing such notification. Furthermore, without adjusting the gas flow rate, only notification that the incomplete combustion has occurred may be performed.

### Fifth Embodiment

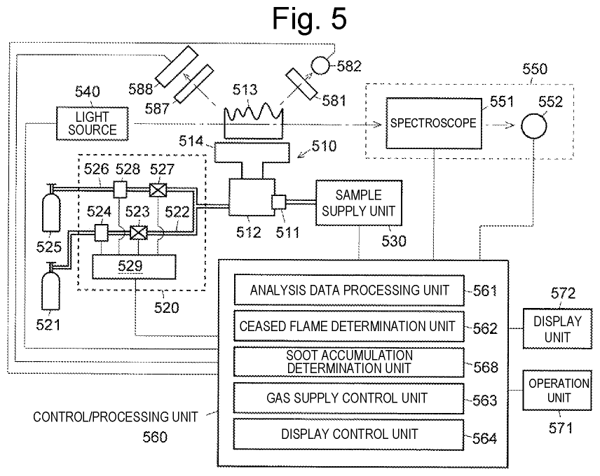

Next, a flame atomic absorption photometer according to the fifth embodiment of the present invention will be described with reference to FIG. 5. FIG. 5 is a configuration diagram of a main part of a flame atomic absorption photometer according to the present embodiment. Note that in the present embodiment, the same or corresponding components as those described in the first embodiment are denoted by the same reference numerals in the last two digits, and the description of such components is appropriately omitted.

The flame atomic absorption photometer according to the present embodiment includes, in addition to the same configuration as the flame atomic absorption photometer according to the first embodiment, a burner head photographing unit 588 that photographs a burner head 514, a bright flame light transmitting bandpass filter 587 disposed between the burner head photographing unit 588 and the burner head 514, and a soot accumulation determination unit 568 that is a functional block provided in a control/processing unit 560.

In a burner 510, when the combustion state of a flame 513 becomes unstable temporarily or continuously, soot is generated and accumulates on the burner head 514. This soot accumulated on the burner head 514 is a cause of impairing the stability of the flame, and thus needs to be appropriately removed. The flame atomic absorption photometer according to the present embodiment has a function of monitoring the accumulation status of soot on the burner head 514.

The burner head photographing unit 588 is an image sensor including a plurality of light detection elements arranged in a two-dimensional matrix. It is desirable that the burner head photographing unit 588 be disposed so as to be able to photograph the entire burner head 514, but the present invention is not limited to this, and the burner head photographing unit may be disposed so as to be able to photograph only the peripheral portion of the slit opening where soot is likely to be accumulated. Note that in FIG. 5, for convenience of drawing, the burner head photographing unit 588 is disposed obliquely above the flame 513, but the position where the burner head photographing unit 588 is provided is not limited to this. The bright flame light transmitting bandpass filter 587 is a bandpass filter that selectively transmits light (bright flame) radiated from soot heated to a high temperature, and selectively transmits light in a wavelength range similar to that of the bright flame light transmitting bandpass filter 485 in the fourth embodiment.

In the flame atomic absorption photometer according to the present embodiment, a detection signal from each light detection element of the burner head photographing unit 588 is amplified by an amplifier (not illustrated), converted into a digital signal by an A/D converter (not illustrated), and input to the soot accumulation determination unit 568 of the control/processing unit 560. On the other hand, the detection signal from the OH-derived light detecting optical sensor 582 is amplified and digitally converted as in the first embodiment, and then input to the control/processing unit 560 (this signal is hereinafter referred to as “OH-derived light detection signal”). The OH-derived light detection signal is input to a ceased flame determination unit 562 as in the first embodiment and is used for determining whether or not the flame 513 has ceased, and is also input to the soot accumulation determination unit 568. The soot accumulation determination unit 568 divides the intensity of the detection signal from each of the light detection elements by the intensity of the OH-derived light detection signal (that is, obtains a ratio of the detection signal from each of the light detection elements to the intensity of the OH-derived light detection signal), and compares the value with a predetermined threshold value T₅. Note that the threshold value T₅ and a predetermined number N may be set at a stage before the present device is delivered to the user or at a stage of installation of the present device, or may be set by the user. Then, among the plurality of light detection elements provided in the burner head photographing unit 588, when the number of light detection elements in which the value obtained by dividing the intensity of the detection signal by the intensity of the OH-derived light detection signal exceeds the threshold value is equal to or more than a predetermined number N (N is an integer of 1 or more), the soot accumulation determination unit 568 determines that soot is accumulated on the burner head 514. In this manner, by determining the accumulation of soot based on the ratio of the detection signal from each light detection element to the intensity of the OH-derived light detection signal, it is possible to cancel a change in the intensity of the bright flame light due to the fluctuation of the flame 513 and to perform accurate determination.

When the soot accumulation determination unit 568 determines that soot is accumulated on the burner head 514, a display control unit 564 controls a display unit 572 to display a predetermined message on the screen, thereby notifying the user that soot is accumulated on the burner head 514. The display control unit 564 and the display unit 572 correspond to a notification unit in the present invention. Note that in addition to or instead of the message, an image representing a region where soot is accumulated on the burner head 514 may be displayed on the screen of the display unit 572. In this case, the soot accumulation determination unit 568 specifies one or more light detection elements among the plurality of light detection elements for which a value obtained by dividing the detection signal by the OH-derived light detection signal exceeds the threshold value T₅, and specifies the radiation position of the bright flame on the burner head 514 (that is, the position where the soot is accumulated) based on the position of the light detection element on the burner head photographing unit 588. Then, under the control of the display control unit 564, an image of the burner head (or an illustration representing the burner head) photographed in advance is displayed on the screen of the display unit 572, and a specific color or a predetermined figure (for example, a line surrounding the region, and the like) is added to a region corresponding to an accumulation position of soot on the image, thereby displaying an accumulation region of soot on the burner head 514. Note that, in this case, the soot accumulation determination unit 568, the display control unit 564, and the display unit 572 correspond to a soot accumulation region presentation unit in the present invention.

Although the embodiment of the present invention is described with specific examples, the present invention is not limited to such an embodiment, and an appropriate change in the scope of the present invention is acceptable.

For example, after the flame 113 is turned on, the flame atomic absorption photometer according to the first to fifth embodiments described above always monitors whether or not an abnormality related to stability of a combustion state of the flame, such as cessation, a sign of backfire, incomplete combustion, or accumulation of soot of the flame 113 has occurred, and notifies the user when it is determined that there is an abnormality. Alternatively, the flame atomic absorption photometer may determine whether or not the abnormality has occurred at a timing instructed by the user or a timing set in advance, and notify the user of a determination result regardless of the result.

Furthermore, in the first to fourth embodiments described above, the bandpass filter (that is, the OH-derived light transmitting bandpass filters 181, 281, 381, and 481, the C₂-derived light transmitting bandpass filters 283 and 383, or the bright flame light transmitting bandpass filter 485) and the optical sensor (that is, the OH-derived light detecting optical sensors 182, 282, 382, and 482, and the C₂-derived light detecting optical sensors 284 and 384, or the bright flame light detection optical sensor 486) that detects light passing through the bandpass filter are provided in the vicinity of the flame forming region. However, the bandpass filter and the optical sensor may not be provided, and the spectroscopes 151, 251, 351, and 451 and the photodetectors 152, 252, 352, and 452 provided in the spectroscopic units 150, 250, 350, and 450 may also serve as the bandpass filter and the optical sensor. In this case, from among the light incident on the spectroscopic units 150, 250, 350, and 450, a wavelength similar to the wavelength transmitted through the above-described OH-derived light transmitting bandpass filters 181, 281, 381, and 481 is selected and guided respectively by the spectroscopes 151, 251, 351, and 451 to the photodetectors 152, 252, 352, and 452, and the detection signals (OH-derived light detection signals) of the photodetectors 152, 252, 352, and 452 at that time are respectively input to the ceased flame determination units 162, 262, 362, and 462, thereby making it possible to determine whether or not the combustion of the flames 113, 213, 313, and 413 is maintained (that is, whether the flame has not ceased). Furthermore, from among the light incident on the spectroscopic units 250 and 350, a wavelength similar to the wavelength transmitted through the above-described C₂-derived light transmitting bandpass filters 283 and 383 is selected and guided respectively by the spectroscopes 251 and 351 to the photodetectors 252 and 352, and the detection signals (C₂-derived light detection signals) of the photodetectors 252 and 352 at that time and the OH-derived light detection signal are respectively input to the backfire sign determination units 265 and 365, thereby making it possible to determine whether or not there is a sign of backfire. Alternatively, from among the light incident on the spectroscopic unit 450, a wavelength similar to the wavelength transmitted through the above-described bright flame light transmitting bandpass filter 485 is selected and guided by the spectroscope 451 to the photodetector 452, and the detection signal (bright flame detection signal) of the photodetector 452 at that time and the OH-derived light detection signal are input to the incomplete combustion determination unit 467, thereby making it possible to determine whether or not incomplete combustion has occurred. Note that in these cases, the light detection for determining the presence or absence of abnormality as described above is performed at a timing different from the light detection for sample analysis. Specifically, for example, after the flames 113, 213, 313, and 413 are turned on, it is determined whether or not the flames 113, 213, 313, and 413 have ceased in a state where no sample is supplied from the sample supply units 130, 230, 330, 430 to the burners 110, 210, 310, and 410, and when it is determined that no cessation has occurred (that is, the combustion of the flames 113, 213, 313, and 413 is maintained), it is further determined whether or not there is a sign of backfire, it is determined whether or not incomplete combustion has occurred, or executes both determinations. When no abnormality has occurred as a result of all the determinations, the sample is supplied from the sample supply units 130, 230, 330, and 430 to the burners 110, 210, 310, and 410 to analyze the sample.

Furthermore, the flame atomic absorption photometer according to the second to fifth embodiments may not include the ceased flame determination units 162, 262, 362, and 462, and the OH-derived light detection signal may be used only to determine the presence or absence of a backfire sign, the presence or absence of incomplete combustion, or the presence or absence of soot accumulation.

Furthermore, the flame atomic absorption photometer according to the present invention may have two or more of a function of determining whether there is a sign of backfire, a function of determining whether incomplete combustion has occurred, and a function of determining whether soot is accumulated.

### Modes

A person skilled in the art would understand that the above-described illustrative embodiments are specific examples of the following modes of the present invention.
(Clause 1) A flame atomic absorption photometer according to Clause 1 includes:
   a burner configured to form a flame by burning a mixture of a mixed gas of a fuel gas and a combustion-assisting gas and a nebulized sample liquid;
   a flame light detection unit configured to detect light radiated from the flame; and
   a ceased flame determination unit configured to determine that the flame has ceased when the intensity of the light detected by the flame light detection unit is lower than a predetermined threshold value; wherein
      the flame light detection unit is configured to selectively detect light having a wavelength of 290 nm or more and 330 nm or less.

With the flame atomic absorption photometer according to Clause 1, it is possible to reliably determine whether or not flame continues to burn normally without being affected by stray light.
(Clause 2) A flame atomic absorption photometer according to Clause 2 includes:
   a burner configured to form a flame by burning a mixture of a mixed gas of a fuel gas and a combustion-assisting gas and a nebulized sample liquid;
   a flame light detection unit configured to detect light radiated from the flame and selectively detect light having a wavelength of 290 nm or more and 330 nm or less;
   a swan band light detection unit configured to selectively detect light of a C₂ swan band among light radiated from the flame; and
   a backfire sign determination unit configured to determine that there is a sign of backfire when a ratio of a light intensity detected by the swan-band light detection unit to a light intensity detected by the flame light detection unit falls below a predetermined threshold value.

With the flame atomic absorption photometer according to Clause 2, the occurrence of backfire can be detected in advance.
(Clause 3) A flame atomic absorption photometer according to Clause 3 is the flame atomic absorption photometer according to Clause 2, further including:
   a gas supply unit configured to supply the fuel gas and the combustion-assisting gas to the burner; and
   a backfire avoidance unit configured to control the gas supply unit so as to reduce a ratio of a flow rate of the combustion-assisting gas to a flow rate of the fuel gas when the backfire sign determination unit determines that there is a sign of backfire.
(Clause 4) A flame atomic absorption photometer according to Clause 4 is the flame atomic absorption photometer according to Clause 2 or 3, further including:
   an exhaust introduction unit configured to introduce a part of exhaust gas generated from the burner into the burner when the backfire sign determination unit determines that there is a sign of backfire.

With the flame atomic absorption photometer according to Clause 3 or Clause 4, the occurrence of backfire can be automatically avoided.
(Clause 5) A flame atomic absorption photometer according to Clause 5 includes:
   a burner configured to form a flame by burning a mixture of a mixed gas of a fuel gas and a combustion-assisting gas and a nebulized sample liquid;
   a flame light detection unit configured to detect light radiated from the flame and selectively detect light having a wavelength of 290 nm or more and 330 nm or less;
   a bright flame detection unit configured to selectively detect light having a wavelength of 800 nm or more and 1100 nm or less among the light radiated from the flame; and
   an incomplete combustion determination unit configured to determine that incomplete combustion has occurred when a ratio of a light intensity detected by the bright flame detection unit to a light intensity detected by the flame light detection unit exceeds a predetermined threshold value.

With the flame atomic absorption photometer according to Clause 5, occurrence of incomplete combustion can be reliably detected.
(Clause 6) A flame atomic absorption photometer according to Clause 6 is the flame atomic absorption photometer according to Clause 5, further including:
   a gas supply unit configured to supply the fuel gas and the supporting gas to the burner; and
   an incomplete combustion cancellation unit configured to control the gas supply unit so as to increase a ratio of a flow rate of the combustion-assisting gas to a flow rate of the fuel gas when the incomplete combustion determination unit determines that incomplete combustion has occurred.

With the flame atomic absorption photometer according to Clause 6, incomplete combustion can be automatically canceled.
(Clause 7) A flame atomic absorption photometer according to Clause 7 includes:
   a burner configured to form a flame by burning a mixture of a mixed gas of a fuel gas and a combustion-assisting gas and a nebulized sample liquid;
   a flame light detection unit configured to detect light radiated from the flame and selectively detect light having a wavelength of 290 nm or more and 330 nm or less;
   a bandpass filter configured to selectively transmit light having a wavelength of 800 nm or more and 1100 nm or less;
   an image sensor including a plurality of light detection elements arranged two-dimensionally and configured to receive light radiated from the burner and passing through the bandpass filter;
   a soot accumulation determination unit configured to determine that soot is accumulated on the burner, by obtaining, for each of the plurality of light detection elements, a ratio of a light intensity detected by the light detection element to a light intensity detected by the flame light detection unit, and when a predetermined number or more of the plurality of light detection elements have the ratio exceeding a predetermined threshold value; and
   a notification unit configured to, when the soot accumulation determination unit determines that soot is accumulated on the burner, notify a user of the determination.

With the flame atomic absorption photometer according to Clause 7, the user can reliably grasp the accumulation status of soot on the burner.
(Clause 8) A flame atomic absorption photometer according to Clause 8 is the flame atomic absorption photometer according to Clause 7, further including:
   a soot accumulation region presentation unit configured to present, to a user, a region on the burner corresponding to one of the plurality of light detection elements having the ratio exceeding a predetermined threshold value as a region where soot is accumulated.

With the flame atomic absorption photometer according to Clause 8, the user can easily grasp the region where soot is accumulated on the burner.

### REFERENCE SIGNS LIST

110 . . . Burner
120 . . . Gas Supply Unit
130 . . . Sample Supply Unit
140 . . . Light Source
150 . . . Spectroscopic Unit
151 . . . Spectroscope
152 . . . Photodetector
160 . . . Control/Processing Unit
162 . . . Ceased Flame Determination Unit
163 . . . Gas Supply Control Unit
164 . . . Display Control Unit
172 . . . Display Unit
181 . . . OH-derived Light Transmitting Bandpass Filter
182 . . . OH-derived Light Detecting Optical Sensor

1. A flame atomic absorption photometer comprising:
a burner configured to form a flame by burning a mixture of a mixed gas of a fuel gas and a combustion-assisting gas and a nebulized sample liquid;
a flame light detection unit configured to detect light radiated from the flame; and
a ceased flame determination unit configured to determine that the flame has ceased when the intensity of the light detected by the flame light detection unit is lower than a predetermined threshold value; wherein
the flame light detection unit is configured to selectively detect light having a wavelength of 290 nm or more and 330 nm or less.

2. A flame atomic absorption photometer comprising:
a burner configured to form a flame by burning a mixture of a mixed gas of a fuel gas and a combustion-assisting gas and a nebulized sample liquid;
a flame light detection unit configured to detect light radiated from the flame and selectively detect light having a wavelength of 290 nm or more and 330 nm or less;
a swan band light detection unit configured to selectively detect light of a C₂ swan band among light radiated from the flame; and
a backfire sign determination unit configured to determine that there is a sign of backfire when a ratio of a light intensity detected by the swan-band light detection unit to a light intensity detected by the flame light detection unit falls below a predetermined threshold value.

3. The flame atomic absorption photometer according to claim 2 further comprising:
a gas supply unit configured to supply the fuel gas and the combustion-assisting gas to the burner; and
a backfire avoidance unit configured to control the gas supply unit so as to reduce a ratio of a flow rate of the combustion-assisting gas to a flow rate of the fuel gas when the backfire sign determination unit determines that there is a sign of backfire.

4. The flame atomic absorption photometer according to claim 2, further comprising:
an exhaust introduction unit configured to introduce a part of exhaust gas generated from the burner into the burner when the backfire sign determination unit determines that there is a sign of backfire.

5. A flame atomic absorption photometer comprising:
a burner configured to form a flame by burning a mixture of a mixed gas of a fuel gas and a combustion-assisting gas and a nebulized sample liquid;
a flame light detection unit configured to detect light radiated from the flame and selectively detect light having a wavelength of 290 nm or more and 330 nm or less;
a bright flame detection unit configured to selectively detect light having a wavelength of 800 nm or more and 1100 nm or less among the light radiated from the flame; and
an incomplete combustion determination unit configured to determine that incomplete combustion has occurred when a ratio of a light intensity detected by the bright flame detection unit to a light intensity detected by the flame light detection unit exceeds a predetermined threshold value.

6. The flame atomic absorption photometer according to claim 5, further comprising:
a gas supply unit configured to supply the fuel gas and the supporting gas to the burner; and
an incomplete combustion cancellation unit configured to control the gas supply unit so as to increase a ratio of a flow rate of the combustion-assisting gas to a flow rate of the fuel gas when the incomplete combustion determination unit determines that incomplete combustion has occurred.

7. A flame atomic absorption photometer comprising:
a burner configured to form a flame by burning a mixture of a mixed gas of a fuel gas and a combustion-assisting gas and a nebulized sample liquid;
a flame light detection unit configured to detect light radiated from the flame and selectively detect light having a wavelength of 290 nm or more and 330 nm or less;
a bandpass filter configured to selectively transmit light having a wavelength of 800 nm or more and 1100 nm or less;
an image sensor including a plurality of light detection elements arranged two-dimensionally and configured to receive light radiated from the flameburner and passing through the bandpass filter;
a soot accumulation determination unit configured to determine that soot is accumulated on the burner, by obtaining, for each of the plurality of light detection elements, a ratio of a light intensity detected by the light detection element to a light intensity detected by the flame light detection unit, and when a predetermined number or more of the plurality of light detection elements have the ratio exceeding a predetermined threshold value; and
a notification unit configured to, when the soot accumulation determination unit determines that soot is accumulated on the burner, notify a user of the determination.

8. The flame atomic absorption photometer according to claim 7 further comprising:
a soot accumulation region presentation unit configured to present, to a user, a region on the burner corresponding to one of the plurality of light detection elements having the ratio exceeding a predetermined threshold value as a region where soot is accumulated.
